(19) United States
(12) Patent Application Publication
(10) Pub. No.: US 2026/0209169 A1
(43) Pub. Date: Jul. 23, 2026

(54) NEW PROCESS FOR THE MANUFACTURE OF MDM2-P53 ANTAGONISTS

(71) Applicant: Boehringer Ingelheim International GmbH, Ingelheim am Rhein (DE)

(72) Inventors: Jidong SHAO, Suhou City (CN); Qing XIAO, Shanghai (CN); Dihan ZHANG, Shanghai (CN); Zhibin ZHU, Shanghai (CN)

(21) Appl. No.: 19/140,403

(22) PCT Filed: Dec. 21, 2023
(86) PCT No.: PCT/EP2023/087179

§ 371 Date: Jun. 18, 2025

(30) Foreign Application Priority Data
Dec. 23, 2022 (WO) PCT/CN2022/141521

(51) Int. Cl. C07C 227/18; C07C 229/66; C07C 249/08; C07C 251/38; C07D 209/38

(52) U.S. Cl. CPC C07C 227/18; C07C 229/66; C07C 249/08; C07C 251/38; C07D 209/38

(57) ABSTRACT

This invention relates to a novel process for the synthesis of substituted aniline of formula (1) or salts thereof via hydroxy-indolinone of formula (13). The process of this invention is palladium free and uses oxygen as green and save oxidant for the copper promoted ring-opening of a hydroxy-indolinone bicycle to a tetra substituted aniline.

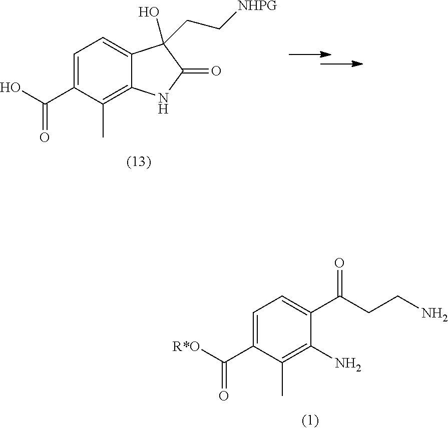

### TECHNICAL FIELD

The present invention relates to efficient and sustainable methods for preparing compounds, which are useful as intermediates in the synthesis of MDM2-p53 antagonists.

### BACKGROUND INFORMATION

Aniline of formula (1) is a consecutive tetra-substituted aromatic compound,

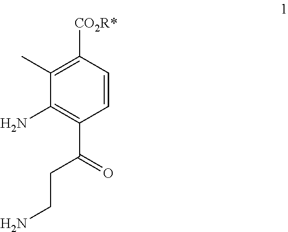

which is used as building block for more complex chemical structures like the structural challenging MDM2-p53 antagonists as described in WO 2017/060431. MDM2-p53 antagonists offer an important approach towards cancer therapy, either as a single agent, or in combination with a broad variety of anti-tumour therapies and thus, there is the need for an efficient and sustainable supply of these compounds.

The previous synthesis as depicted in scheme 1 (with R*=methyl, see WO 2017/060431) employs three steps relying on homogeneous palladium catalysis. This reaction sequence starts with an ortho-selective bromination as described in Angew. Chem. Int. Ed. 2011, 50, 5524-5527 to generate phenyl bromide of formula (3) using up to 5 mol % Pd(OAc)₂. Followed by a Sonogashira coupling, Chem. Rev. 2007, 107, 874, of the phenyl bromide of formula (3) with a terminal alkyne of formula (4) to generate a compound of formula (5). The Sonogashira coupling employs a palladium catalyst as well as a copper co-catalyst to form the carbon-carbon bond, otherwise the reaction conditions are relatively mild. In a third palladium catalysed step the alkyne is hydrated assisted by the oxygen of the neighbouring group amide, J. Org. Chem., 2015, 80, 7594, to a compound of formula (6).

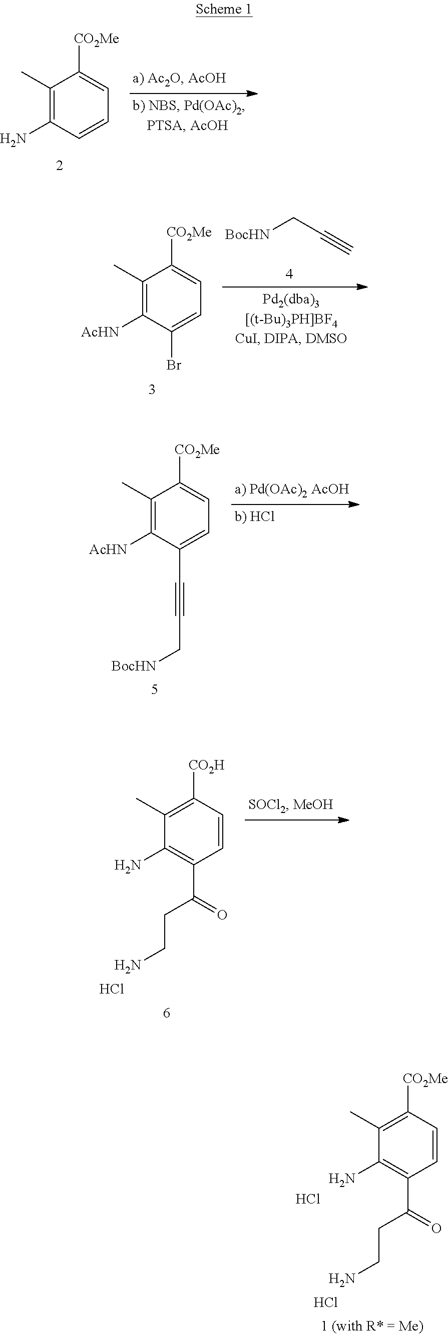

This leads not only to economic and ecological issues but also to the challenge of reducing the heavy metal palladium in the product to low residue levels, which correspond to the strict limits necessary for a medicinal product for administration to humans.

Therefore, there is the need to develop a palladium free process to intermediates like the highly substituted aniline of compound of formula (1) for the synthesis of MDM2-p53 antagonists.

### BRIEF SUMMARY OF THE INVENTION

This invention relates to a novel process for the synthesis of substituted anilines of formula (1) via isatin derived compound of formula (13). Especially the consecutive tetra-substitution of the aromatic ring present in compounds of formula (14) and (1) is a synthetic challenge. The process of this invention is palladium free and uses oxygen as green and save oxidant for the copper promoted ring-opening of the hydroxy-indolinone of formula (13).

### DETAILED DESCRIPTION OF THE INVENTION

This invention relates to a process for the manufacturing of an aniline of formula (1) wherein the substitution pattern of the aniline of formula (1) is derived from an isatin of formula (9).

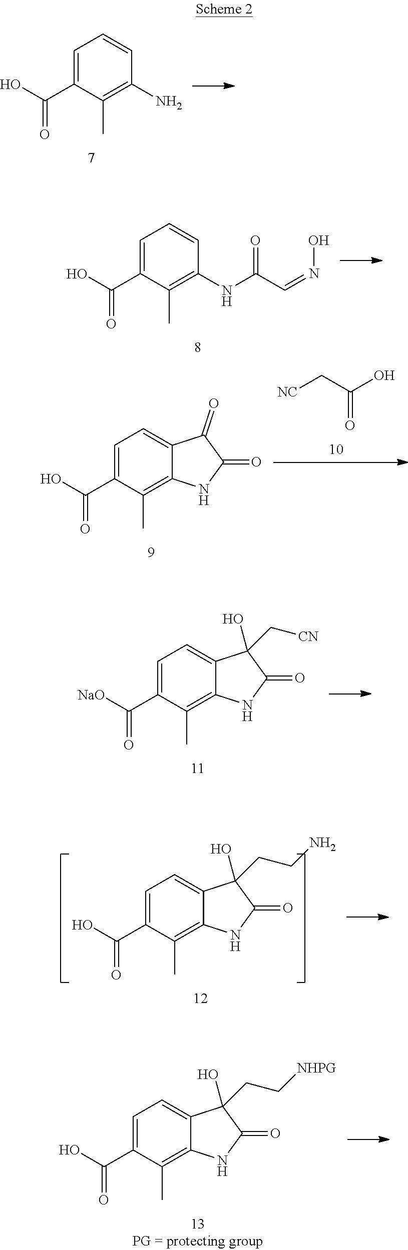

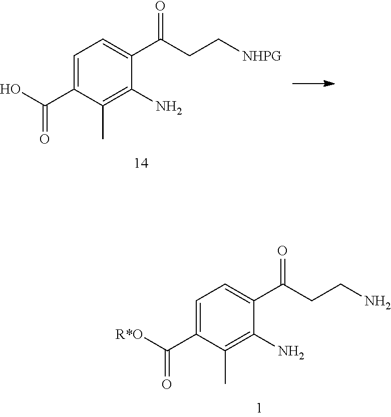

In a first aspect this invention relates to a process for the manufacturing of compounds of formula (14) or (1) comprising an oxidative ring-opening of the hydroxy-indolinone of the formula (13). PG stands for a protective group; possible amino protective groups are known to a person skilled in the art. Any amino protective group as described in T. W. Greene & P. G. M Wuts, “Protective Groups in Organic Synthesis,” 4th Edition, John Wiley & Sons, Inc. (2007), can be used. Preferably the protective group is selected from a group comprising an electron withdrawing group —C(═O)—R, wherein R is R′ or —O—R′ and R′ stands for linear, branched, or cyclic aliphatic or aromatic groups or combinations thereof, optionally substituted. Preferably, the PG used is Ac or Boc. R* stands for a cleavable carboxyl protecting group. R* is preferably an optionally substituted C₁₋₆alkyl or an optionally substituted benzyl group. Most preferred R* is methyl, ethyl, iso-propyl or benzyl.

In literature K₃[Fe(CN)₆] was reported in CN102675125 and t-BuOOH (3.0 eq.) was reis ported in Org. Lett. 2017, 19, 5, 988 as oxidants in the presence of KOH to form aniline products.

Thus, in one aspect of the invention the oxidative ring-opening of the compound of formula (13) comprises the use of a Fe oxidant. In one embodiment the Fe oxidant used in the oxidative ring opening is K₃[Fe(CN)₆], preferably used together with KOH as a base. When K₃[Fe(CN)₆] was used as an oxidant, it was found that preferably at least 1 equivalent KOH should be used for ring opening. Additionally, it was found, that the preferred solvent system is a mixture of MeCN/H₂O.

Conversion of compound of formula (13) to product of formula (14) as described above comprising Fe oxidant gave moderate to good yields thus providing a cost-efficient alternative towards the tetra substituted aniline.

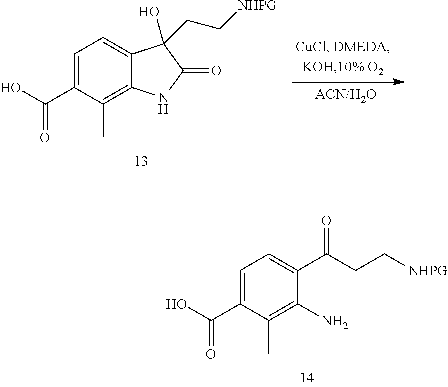

In an alternative approach, it was surprisingly found that the use of a Cu catalyst for the oxidative ring opening of a compound of formula (13) gave most promising results. The Cu catalyst can be selected from the non-exhaustive list of CuI, CuBr, CuCl, CuO, CuCN, CuF, CuNO₃, Cu₂O, CuCl₂, CuSO₄, Cu(NO₃)₂, Cu(OAc)₂ and Cu(acac)₂, preferably CuCl is used.

The amount of CuCl used in the ring opening can be between 0.2 to 1.0 equiv., preferably between 0.4 to 0.8 equiv., most preferably approximately 0.6 equiv.

The ligand can be selected from the non-exhaustive list of 8-hydroxyquinoline 1,10-phenanthroline, dibenzoylmethane, DMEDA, TMEDA, bipyridine, diaminocyclohexane and related diamine or pincer type ligands, preferably DMEDA is used.

The amount of DMEDA used is preferably between 2 to 10 equiv., more preferably between 3 to 6 equiv., most preferably approximately 5 equiv.

The mol ratio Cu:DMEDA can be between 1:2 to 1:5, most preferably 3:10.

The oxidant can be selected from the non-exhaustive list of air, O₂, O₂ mixed with inert gas such as CO₂, argon or N₂, H₂O₂, tBuOOH, and NaIO₄. Preferably, the oxidative ring-opening uses oxygen as stoichiometric oxidant where the oxygen is from air or from a mixture of O₂ in N₂. The concentration of O₂ in N₂ used is preferably between 8 to 21% v/v. For save handling of oxygen and organic vapor 8 to 10% v/v concentration is most preferred. Lower concentration of oxygen will lead to lower reaction rate.

In a further aspect of the invention the compound of formula (13) used for the oxidative ring-opening as described herein is synthesized comprising a catalytic hydrogenation of the nitrile of formula (11). Such catalytic hydrogenation leads to the amine of formula (12) and then to the compound of formula (13) after introduction of the protecting group PG. The catalytic hydrogenation can be achieved using various heterogenous heavy metal catalysts, like Pd/C, Pd/Al₂O₃, PtO₂, Ru/C, Rh/C or spongy nickel. Preferably, spongy nickel is used due to its stability and high catalytic activity at room temperature. Typically, the catalyst can be recycled and reused for the subsequent batch. Additionally, the residue in the product is very low due to the heterogenous nature of the nickel catalyst.

It was surprisingly found that the hydrogenation of the nitrile of formula (11) and protection of amine of formula (12) can be accomplished in a one-pot reaction if the protecting group PG is a Boc protecting group. One-pot reactions have the advantage that multiple transformation can be achieved in a single reaction vessel without isolation or purification which impacts the economy and ecology of the process due to the reduction in time, reagents and solvents needed. In this invention the hydrogenation with spongy nickel can be performed in the presence of the protection reagents and directly converted to Boc carbamate of formula (13).

In a further aspect of the invention the compound of formula (11) used for the catalytic hydrogenation as described herein is synthesized comprising a decarboxylative addition of cyanoacetic acid to isatin of formula (9). Here the base and solvent were optimized for large scale manufacturing. It was found that DMF and THE can be employed as organic solvents, however a mixture of DMF/THF (1V/3V) reduces the amount of undesired side products. The yield can be improved by increasing the amount of TEA from 0.2 to 1.0 equivalents.

In a further aspect of the invention the isatin of formula (9) used for the decarboxylative addition as described herein is synthesized comprising isatin ring formation of the oxime of formula (8). Preferably, the ring formation is conducted with concentrated H₂SO₄, preferably in a temperature range of 90 to 100° C., in high yields. Temperature control is critical as decomposition of compound of formula (9) was observed to start at 120° C.

Finally, in a further aspect of the invention the oxime of formula (8) is synthesized comprising reacting the aniline of formula (7) with chloral hydrate in the presence of hydroxylamine hydrochloride.

Although the isatin formation applied well-known Sandmeyer methodology the reaction conditions had to be intensively investigated due to the substitution pattern of the starting aniline. It was also found that one of the advantages of the use of acid substituted aniline of formula (7) (instead of e.g. corresponding esters) as starting material is the solid state of product of formula (8), which can be used without further purification.

In another alternative aspect of this invention the isatin formation and decarboxylative addition can also be conducted using, e.g., the respective methyl ester analogues of formula (7′) to (11′) instead of the free acids according to Scheme 3.

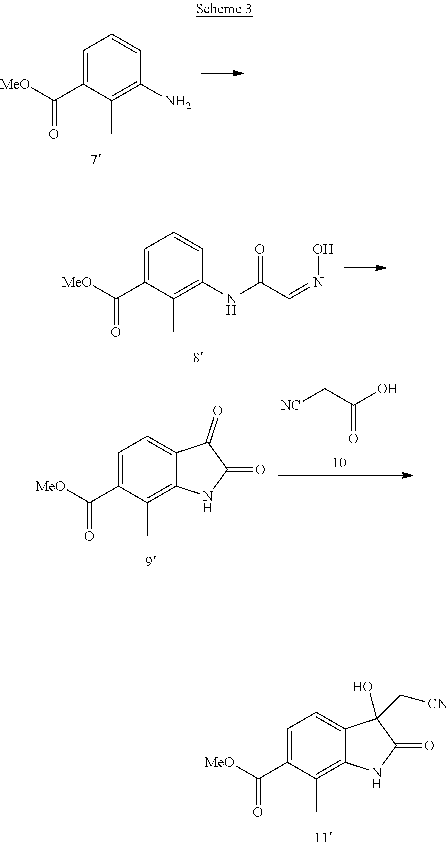 [SMILES: COC(=O)c1cccc(N)c1C ; COC(=O)c1cccc(NC(=O)/C=N\O)c1C ; COC(=O)c1ccc2c(c1C)NC(=O)C2=O ; N#CCC(=O)O ; COC(=O)c1ccc2c(c1C)NC(=O)C2(O)CC#N]

However, a column chromatography is then necessary to purify oxime of formula (8′) for isatin ring closure. Additionally, milder conditions for the isatin ring formation are necessary as otherwise undesired by-products are formed in up to 70% yield. This can be achieved by employing MsOH at lower temperatures.

Another aspect of this invention is the use of the processes as described herein for manufacturing intermediates and starting materials for MDM2-p53 antagonists. Another aspect is the use of the processes as described herein for the synthesis of an MDM2-p53 antagonist.

USED TERMS AND DEFINITIONS

| | |
|---|---|
| Ac | acetyl |
| AcOH | acetic acid |
| ACN | acetonitrile |
| aq. | aquatic, aqueous |
| Boc | tert-butyloxycarbonyl |
| C | concentration |
| d | day(s) |
| dba | dibenzylideneacetone |
| DIPA | diisopriopylamine |
| DMEDA | N,N'-dimethyl-1,2-ethanediamine |
| DMF | N,N-dimethylformamide |
| DMSO | dimethyl sulfoxide |
| equiv. | equivalent(s) |
| ESI | electron spray ionization |
| Et | ethyl |
| h | hour(s) |
| HPLC | high performance liquid chromatography |
| IPAc | isopropyl acetate |
| conc. | concentrated |
| LC | liquid chromatography |
| Me | methyl |
| MeCN | acetonitrile |
| MeOH | methanol |
| min | minute(s) |
| MS | mass spectrometry |
| MsOH | Methane sulfonic acid |
| NBS | N-bromosuccinimide |
| NP | normal phase |
| n.a. | not available |
| PG | protective group |
| Ph | phenyl |
| PTSA | p-toluenesulfonic acid |
| Rf (R_{f}) | retention factor |
| RP | reversed phase |
| rt | ambient temperature |
| S | second(s) |
| TEA | triethyl amine |
| temp. | temperature |
| t/tert | tertiary |
| Tf | triflate |
| THF | tetrahydrofuran |
| TLC | thin layer chromatography |
| t_{Ret.} | retention time (HPLC) |
| HPLC | high performance liquid chromatography |
| UV | ultraviolet |
| wt | weight |

### EXAMPLES

### General

Unless stated otherwise, all the reactions are carried out in commercially obtainable apparatus using methods that are commonly used in chemical laboratories. Starting materials that are sensitive to air and/or moisture are stored under protective gas and corresponding reactions and manipulations therewith are carried out under protective gas (nitrogen or argon).

If a compound is to be represented both by a structural formula and by its nomenclature, in the event of a conflict the structural formula is decisive.

### Chromatography

The analytical HPLC (reaction control) of intermediate and final compounds is carried out using columns made by Waters (names: XBridge™ CSH phenyl Hexyl, 3.5 um, 3×100 mm, 3.5 um) and Agilent (names: Eclipse XDB-C8, 5 μm, 4.6×150 mm).

HPCL method A:

| HPLC | Gradient HPLC apparatus | |
|---|---|---|
| | (Agilent 1260 Series system) | |
| Detection signal | 250 | nm (bandwidth |
| | 4 | nm, reference off) |
| Column | Xselect CSH phenyl Hexyl, | |
| | 3 × 100 | mm, |
| | 3.5 | um, |
| | part No.: 186005372 | |
| Column | 35° | C. |
| temperature | | |
| Solvent | A: | 100% Buffer solution |
| | B: | 100% Acetonitrile (HPLC grade) |
| Buffer solution | Dissolve 770.8 mg NH₄OAc | |
| | (HPLC grade) in 1.0 L HPLC water, | |
| | adjust pH to 3.8 with | |
| | acetic acid (AR grade)) | |
| Flow | 1.2 | mL/min |
| Gradient | 0 min | 5% B |
| | 15 min | 95% B |

HPCL method B:

| HPLC | Gradient HPLC apparatus | |
|---|---|---|
| | (Agilent 1260 Series system) | |
| Detection signal | 225 | nm (bandwidth |
| | 4 | nm, reference off) |
| Column | Xselect CSH phenyl Hexyl, | |
| | 3 × 100 | mm, |
| | 3.5 | um, |
| | part No.: 186005372 | |
| Column | 35° | C. |
| temperature | | |
| Solvent | A: | 100% Buffer solution |
| | B: | 100% Acetonitrile (HPLC grade) |
| Buffer solution | Dissolve 770.8 mg NH₄OAc | |
| | (HPLC grade) in 1.0 L HPLC | |
| | water, adjust pH to 3.8 with | |
| | acetic acid (AR grade)) | |
| Flow | 1.2 | mL/min |
| Gradient | 0 min | 5% B |
| | 15 min | 95% B |

HPCL method C:

| HPLC | Gradient HPLC apparatus | |
|---|---|---|
| | (Agilent 1260 Series system) | |
| Detection signal | 225 | nm (bandwidth |
| | 4 | nm, reference off) |
| Column | Eclipse XDB-C8, | |
| | 4.6 × 150 | mm, |
| | 5 | um, Agilent; |
| | Part No. 993967-906 | |
| Column | 40° | C. |
| temperature | | |
| Solvent | A: Buffer solution/acetonitrile = 9/1, (V/V) | |
| | B: Buffer solution/acetonitrile = 2/8, (V/V) | |
| Buffer solution | Dissolve 1.0 g KH₂PO₄ | |
| | (AR grade) in 1.0 L water, | |
| | adjust pH to 1.7 with | |
| | HClO₄ (AR grade) | |
| Flow | 1.0 | mL/min |
| Gradient | 0 min | 0% B |
| | 2 min | 10% B |
| | 7 min | 60% B |
| | 10 min | 100% B |
| | 12 min | 100% B |

### Example 1

### 3-(2-(Hydroxyimino)acetamido)-2-methylbenzoic acid (8)

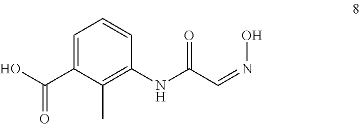 [SMILES: Cc1c(NC(=O)/C=N\O)cccc1C(=O)O]

1973 g (7.0 eq.) Na₂SO₄ are stirred in 3900 g water at 40-50° C. for 1 h. Then 120 g HCl_{conc.} and 300 g (1.0 equiv.) compound 7 are added and the temperature is increased to approximately 55° C. Then 483 g (3.5 equiv.) NH₂OH·HCl and 492 g (1.5 equiv.) chloral hydrate in 1200 g water are added and the mixture is stirred for 5 h at 55° C. Then the temperature is lowered to 40° C. and filtered. The filter cake is washed with 2400 g water and dried under reduced pressure for 72 h to give 380 g compound 8 (86.2% yield in 94.8 A %)¹H NMR (400 MHz, DMSO-d₆) δ 2.35 (s, 3H), 7.29 (dd, J=7.8 Hz, 1H), 7.55 (d, J=7.8 Hz, 1H), 7.62 (dd, J=7.7, 1.3 Hz, 1H), 7.68 (s, 1H), 9.73 (s, 1H), 12.22 (s, 1H), 12.98 (s, 1H). ¹³C NMR (100 MHz, DMSO-d₆) δ 14.99, 125.57, 127.21, 129.08, 132.65, 133.30, 136.49, 143.67, 160.68, 169.03. 3-(2-(Hydroxyimino)acetamido)-2-methylbenzoic acid (8) analysed with HPLC method A, t_{Ret}: 2.05 min.

### Example 2

### 7-Methyl-2,3-dioxoindoline-6-carboxylic acid (9)

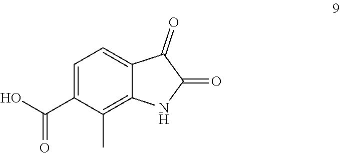

2000 g H₂SO_{4conc.} is heated to 85-90° C. and 370 g compound 8 (1.0 equiv.) are added. The reaction is stirred at approximately 90° C. for 30 min before cooling to 15-25° C. 3100 g water and 440 g acetone are mixed in a second vessel and cooled to 0-10° C. The reaction mixture is transferred to the second reaction vessel while maintaining the temperature below 20° C. After complete addition the reaction mixture is stirred between 10-20° C. for 1 h. The reaction is filtered and the filter cake is washed with 1500 g water and dried under reduced pressure for 72 h to give 224 g of compound 9 (65.6% yield in 83.5 A %). ¹H NMR (400 MHz, DMSO-d₆) δ 2.31 (s, 3H), 7.34-7.43 (m, 2H), 11.22 (s, 1H), 13.34 (s, 1H). ¹³C NMR (100 MHz, DMSO-d₆) δ 13.36, 119.12, 121.28, 121.53, 123.55, 139.95, 149.97, 159.85, 168.14, 184.65. 7-Methyl-2,3-dioxoindoline-6-carboxylic acid (9) analysed with HPLC method A, t_{Ret}: 1.42 min.

### Example 3

### Sodium 3-(cyanomethyl)-3-hydroxy-7-methyl-2-oxoindoline-6-carboxylate (11)

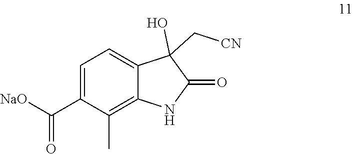

220 g of compound 9 (1.0 equiv.) are added to a mixture 314 g DMF, 370 g THE and 110 g TEA (1.0 equiv.) at a rate to keep the temperature between 15-35° C. Then the reaction is warmed to 60° C. and 99 g (1.1 equiv.) cyanoacetic acid (10) in 98 g THF is added and stirred for 2 h. Then the mixture is cooled to 25-35° C. and 440 g aqueous 10% NaOH is added at a rate to maintain the temperature below 35° C. and stirred for 30 min. The mixture is cooled to 10-20° C. and filtered. The filter cake is washed with 390 g THE and dried under reduced pressure to give 202 g of compound 11 (70.2% yield and 98.9 A %). ¹H NMR (400 MHz, DMSO-d₆) δ 2.30 (s, 3H), 2.91 (d, J=16.6 Hz, 1H), 3.04 (d, J=16.6 Hz, 1H), 6.74 (s, 1H), 7.16 (m, 2H), 10.49 (s, 1H). ¹³C NMR (100 MHz, DMSO-d₆) δ 14.37, 26.15, 72.33, 117.19, 120.14, 120.14, 121.76, 127.78, 140.11, 144.74, 172.59, 177.42. Sodium 3-(cyanomethyl)-3-hydroxy-7-methyl-2-oxoindoline-6-carboxylate (11) analysed with HPLC method B, t_{Ret}: 1.75 min.

### Example 4

### 3-(2-((tert-butoxycarbonyl)amino)ethyl)-3-hydroxy-7-methyl-2-oxoindoline-6-carboxylic acid (13a)

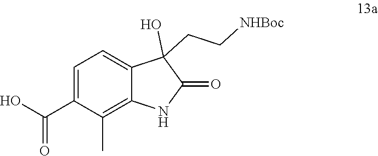 [SMILES: Cc1c(C(=O)O)ccc2c1NC(=O)C2(O)CCNC(=O)OC(C)(C)C]

70 g (1.0 equiv.) of compound 11 and 28 g (1.0 equiv.) Na₂CO₃ are dissolved in 490 g water. 14 g Ni catalyst in 140 g water is added, followed by a solution of 114 g (2.0 equiv.) Boc₂O in 187 g THF. The mixture is purged with N₂ three times and pressurized with 12-14 bar H₂. After stirring at ambient temperature for 12 h H₂ pressure is released, purged with N₂ three times and filtered. The filer cake is washed with 140 g water and the filtrate transferred to a second reactor where 183 g IPAc are added. The mixture is stirred at ambient temperature for 20 min and the aqueous layer collected. The pH of the aqueous layer is adjusted to pH 4.9-5.2 by addition of 57 g HCl_{conc.}, then 0.15 g seeding crystals are added and stirring continued for 1.5 h. Then the pH is adjusted to pH 3.0-4.0 by addition of HCl_{conc.}, and the temperature is cooled to 10-20° C. and stirring continued for 2 h. If the pH remains under 4.0 the crystal product can be collected, otherwise pH adjusted to 4.0 is needed with an additional amount of HCl_{conc.} The filter cake is washed with 140 g water and dried under reduced pressure at approximately 70° C. for at least 16 h to obtain 77 g 13a (80.2% yield and 95.8 A %). ¹H NMR (400 MHz, DMSO-d₆) δ 1.33 (s, 9H), 1.81-1.96 (m, 2H), 2.37 (s, 3H), 2.85 (tdd, J=15.3, 10.0, 5.5 Hz, 2H), 6.04 (s, 1H), 6.69 (t, J=5.5 Hz, 1H), 7.18 (d, J=7.7 Hz, 1H), 7.47 (d, J=7.7 Hz, 1H), 10.47 (s, 1H), 12.86 (s, 1H). ¹³C NMR (100 MHz, DMSO-d₆) δ 14.35, 28.15, 34.81, 37.36, 74.58, 77.46, 119.73, 120.92, 123.91, 131.70, 134.93, 141.16, 155.29, 168.66, 179.24. 3-(2-((tert-butoxycarbonyl)amino)ethyl)-3-hydroxy-7-methyl-2-oxoindoline-6-carboxylic acid (13a) analysed with HPLC method C, t_{Ret}: 5.78 min.

### Example 5

### 3-amino-4-(3-((tert-butoxycarbonyl)amino)propanoyl)-2-methylbenzoic acid (14a)

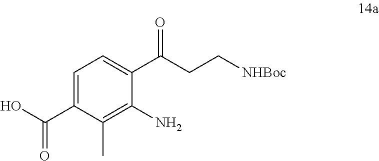 [SMILES: Cc1c(C(=O)O)ccc(C(=O)CCNC(=O)OC(C)(C)C)c1N]

24 g (0.6 equiv.) CuCl and 70 g (2.0 equiv.) DMEDA are added to a solution of 316 g MeCN and 100 g water and stirred for 30 min at ambient temperature. A solution of 140 g (1.0 equiv.) of compound 13a and 67 g (3.0 equiv.) KOH in 300 g water are added. A gas mixture of 10% O₂ in N₂ is passed into the solution at a rate of 50 mL/min for 18 h at 60 70° C. The mixture is cooled to 0-10° C. and 470 g 6N HCl is added to adjust the pH to 2.0-2.5 and stirring is continued at 0-15° C. for 2 h. The mixture is filtered, and the filter cake washed with 280 g water. The cake is transferred to another reaction vessel and dissolved in 400 g MeCN. A solution of 43 g KOH in 500 g water is added and stirred for 2 h at ambient temperature. The mixture is filtered over celite, and the filter cake washed with 280 g water. The filtrate is cooled to 0-10° C. and 135 g 6N HCl are added to adjust the pH to 3.0-4.0 and stirring is continued at 0-15° C. for 2 h. The mixture is filtered, and the filter cake is washed with 280 g water and dried under reduced pressure at approximately 50-60° C. for at least 16 h to obtain 77 g 14a (91.8% yield and 98.0 A %). ¹H NMR (400 MHz, DMSO-d₆) δ 1.37 (s, 9H), 2.21 (s, 3H), 3.09 (s, 1H), 3.12 (s, 1H), 3.26 (td, J=6.5 Hz, 2H), 6.75-6.85 (m, 2H), 7.30 (s, 2H), 7.71 (d, J=8.4 Hz, 1H), 13.13 (s, 1H). ¹³C NMR (100 MHz, DMSO-d₆) δ 13.84, 28.18, 36.17, 36.17, 77.55, 113.94, 116.88, 122.52, 128.72, 128.72, 149.81, 155.48, 169.83, 201.04. 3-amino-4-(3-((tert-butoxycarbonyl)amino)propanoyl)-2-methylbenzoic acid (14a) analysed with HPLC method C, t_{Ret}: 8.40 min.

### Example 6

### Methyl 3-amino-4-(3-aminopropanoyl)-2-methylbenzoate dihydrochloride (1a)

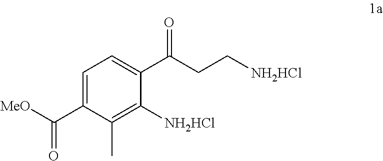

100 g (1.0 equiv.) of 14a is stirred in 950 g MeOH and heated to 50-60° C. Then SOCl₂ (266 g, 6.0 equiv.) is added at such a rate, that the temperature remains between 50-60° C. When the addition is finished the reaction is stirred at 55-65° C. for 1 h. The solvent is removed under reduced pressure to 500-600 mL distillate. The residue is azeotrophically distilled at 55-65° C. with THE in 3 portions of 350 g, 700 g, and 700 g. The reaction is cooled to 10-20° C. and filtered. The filter cake is washed with THE and dried under reduced pressure to give 98 g of 1 (85.6% yield in 98.47 w/w %). ¹H NMR (400 MHz, DMSO-d₆) δ 2.22 (s, 2H), 3.08-3.14 (m, 2H), 3.44 (t, J=8.0 Hz, 2H), 3.85 (s, 3H), 6.85 (d, J=8.5 Hz, 1H), 7.71 (d, J=8.5 Hz, 1H), 8.29 (d, J=6.3 Hz, 3H), 9.25 (s, 3H). ¹³C NMR (101 MHz, DMSO-d₆) δ 13.98, 34.25, 36.38, 52.26, 114.35, 116.92, 123.46, 128.46, 136.01, 149.39, 168.27, 199.25. Methyl 3-amino-4-(3-aminopropanoyl)-2-methylbenzoate dihydrochloride (1a) analysed with HPLC method C, t_{Ret}: 5.91 min.

1. A process for the manufacturing of a compound of formula (1) 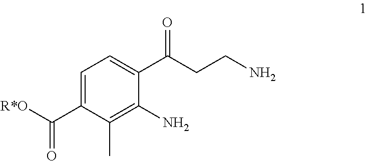
or a salt thereof,
comprising the oxidative ring-opening of a compound of formula (13) 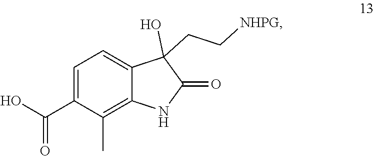
wherein
R* is a cleavable carboxyl protecting group, preferably an optionally substituted C₁₋₆alkyl or an optionally substituted benzyl group, most preferably selected from the group consisting of methyl, ethyl, iso-propyl and benzyl, and
PG is a suitable amino protective group, preferably a group —C(═O)—R, wherein R is R′ or —O—R′ and R′ is an optionally substituted linear, branched or cyclic aliphatic or aromatic group or combinations thereof, most preferably a protective group selected from the group consisting of acetyl (Ac) and tert-butyloxy carbonyl (Boc).

2. The process according to claim 1, wherein the oxidative ring-opening uses a Fe oxidant.

3. The process according to claim 2, wherein the oxidant is K₃[Fe(CN)₆].

4. The process according to claim 3, wherein KOH is used as base.

5. The process according to claim 1, wherein the oxidative ring-opening uses a Cu catalyst.

6. The process according to claim 5, wherein the oxidative ring-opening uses oxygen as stoichiometric oxidant.

7. The process according to claim 6, wherein the oxygen used is in a concentration of O₂ in N₂ between 8 to 21% v/v, preferably between 8 to 10% v/v.

8. The process according to claim 1, wherein the compound of formula (13) is synthesized comprising a catalytic hydrogenation of a compound of formula (11)

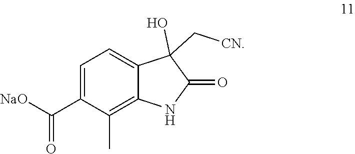

9. The process according to claim 8, wherein the catalytic hydrogenation of the compound of formula (11) is catalysed with spongy nickel.

10. The process according to claim 8, wherein the compound of formula (11) is synthesized comprising a decarboxylative addition of cyanoacetic acid to the compound of formula (9)

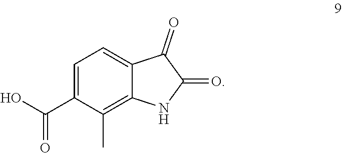

11. The process according to claim 10, wherein the compound of formula (9) is synthesized comprising isatin ring formation of the compound of formula (8)

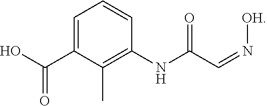 [SMILES: Cc1c(NC(=O)/C=N\O)cccc1C(=O)O]

12. The process according to claim 11, wherein the isatin ring formation is conducted with concentrated H₂SO₄.

13. The process according to claim 11, wherein the compound of formula (8) is synthesized comprising reacting a compound of formula (7) 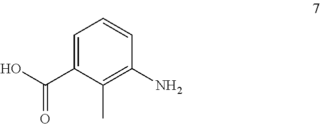
with chloral hydrate in the presence of hydroxylamine hydrochloride.

14. The use of a process according to claim 1 for the synthesis of an MDM2-p53 antagonist.
